(19) United States
(12) Patent Application Publication
(10) Pub. No.: US 2016/0089271 A1
(43) Pub. Date: Mar. 31, 2016

(54) Method for Alignment of Intraocular Lens

(71) Applicant: Jaime Zacharias, Santiago (CL)

(72) Inventor: Jaime Zacharias, Santiago (CL)

(21) Appl. No.: 14/496,147

(22) Filed: Sep. 25, 2014

(51) Int. Cl. A61F 9/008

(52) U.S. Cl. CPC A61F 9/00834; A61F 2/1662

(57) ABSTRACT

A method for precise intraocular delivery of an astigmatic intraocular lens in a patient's eye includes recording traceable eye landmarks, recording the corneal astigmatism, registering the recorded astigmatism axis to the recorded traceable eye landmarks, providing a light source for generating a light beam, providing a scanner for deflecting the light beam to form an enclosed treatment pattern that includes a visible registration feature, providing a delivery system that delivers the enclosed treatment pattern to target tissue in the patient's eye to form an enclosed incision therein including the visible registration feature linkable to the recorded traceable eye landmarks registered to the corneal astigmatism axis. Inserting an intraocular lens within the enclosed incision, wherein the intraocular lens has an astigmatism axis registration feature visible to the surgeon to align with the patient's eye visible astigmatism axis registration feature of the enclosed incision.

### RELATED APPLICATIONS

This application is related to Patent Applications US20100137982, US20110202046 and US20110184395 which are incorporated herein by reference.

### FIELD OF THE INVENTION

The present invention relates to ophthalmic surgical procedures and systems.

### BACKGROUND OF THE INVENTION

Cataract extraction is one of the most commonly performed surgical procedures in the world with estimated 2.5 million cases performed annually in the United States and 9.1 million cases worldwide in 2000. This was expected to increase to approximately 13.3 million estimated global cases in 2006. This market is composed of various segments including intraocular lenses for implantation, viscoelastic polymers to facilitate surgical maneuvers, disposable instrumentation including ultrasonic phacoemulsification tips, tubing, and various knives and forceps. Modern cataract surgery is typically performed using a technique termed phacoemulsification in which an ultrasonic tip with an associated water stream for cooling purposes is used to sculpt the relatively hard nucleus of the lens after performance of an opening in the anterior lens capsule termed anterior capsulotomy or more recently capsulorhexis. Following these steps as well as removal of residual softer lens cortex by aspiration methods without fragmentation, a synthetic foldable intraocular lens (IOL) is inserted into the eye through a small incision.

One of the earliest and most critical steps in the procedure is the performance of capsulorhexis. This step evolved from an earlier technique termed can-opener capsulotomy in which a sharp needle was used to perforate the anterior lens capsule in a circular fashion followed by the removal of a circular fragment of lens capsule typically in the range of 5-8 mm in diameter. This facilitated the next step of nuclear sculpting by phacoemulsification. Due to a variety of complications associated with the initial can-opener technique, attempts were made by leading experts in the field to develop a better technique for removal of the anterior lens capsule preceding the emulsification step. The concept of the capsulorhexis is to provide a smooth continuous circular opening through which not only the phacoemulsification of the nucleus can be performed safely and easily, but also for easy insertion of the intraocular lens. It provides both a clear central access for insertion, a permanent aperture for transmission of the image to the retina by the patient, and also a support of the IOL inside the remaining capsule that would limit the potential for dislocation.

Using the older technique of can-opener capsulotomy, or even with the continuous capsulorhexis, problems may develop related to inability of the surgeon to adequately visualize the capsule due to lack of red reflex, to grasp it with sufficient security, to tear a smooth circular opening of the appropriate size without radial rips and extensions or technical difficulties related to maintenance of the anterior chamber depth after initial opening, small size of the pupil, or the absence of a red reflex due to the lens opacity. Some of the problems with visualization have been minimized through the use of dyes such as methylene blue or indocyanine green. Additional complications arise in patients with weak zonules (typically older patients) and very young children that have very soft and elastic capsules, which are very difficult to mechanically rupture.

Many cataract patients are astigmatic. Astigmatism can occur when the cornea has a different curvature one direction than the other. Both the anterior and posterior surfaces of the cornea can contribute to total corneal astigmatism. The anterior surface is usually considered for calculation although new instruments are being designed to measure both surfaces for improved accuracy. Toric IOLS are used for correcting astigmatism but require precise placement, orientation, and stability. Other means for correction often involve making the corneal shape more spherical, or at least more radially symmetrical. There have been numerous approaches, including Corneoplasty, Astigmatic Keratotomy (AK), Corneal Relaxing Incisions (CRI), and Limbal Relaxing Incisions (LRI). All are done using manual, mechanical incisions. Presently, astigmatism cannot easily or predictably be fully corrected. About one third of those who have surgery to correct the irregularity find that their eyes regress to a considerable degree and only a small improvement is noted. Another third find that the astigmatism has been significantly reduced but not fully corrected. The remaining third have the most encouraging results with the most or all of the desired correction achieved.

Femtosecond Laser based methods to aid in the precise alignment of astigmatic IOLs have been proposed, such as in US20100137982 Patent Application. While these methods may be usable, they rely on the added requirement of specially designed intraocular lenses with protrusions or extensions that may not be approved for human use until extensive safety studies are performed. Also, they rely on adding a complex step to the surgery where the surgeon needs to match, engage and interlock capsule incision features with IOL features.

What is needed are ophthalmic methods, techniques and apparatus to advance the standard of care of the astigmatic cataract patient while using the installed base of astigmatic intraocular lenses and maintaining the conventional surgical implantation procedure of the same.

### PRIOR ART

Prior art technologies for astigmatism alignment have consisted in placement of alignment ink marks by the surgeon on the eye surface, based on pre-operative astigmatism measurements, and more recently, operating microscope video overlay systems (SMI Surgical Guidance, Senso-Motoric Instruments, Germany; Callisto-Z Align, Carl Zeiss, Germany). These systems are expensive as they are based on complex real-time eye feature tracking and heads-on displays or light marks projection.

A different proposal for IOL astigmatism axis alignment has been described in Patent Application No. US20100137982. This proposal has the disadvantage that it strictly depends on availability of a specially designed, compatible IOLs for the proposed method. These capsular incision matching IOLs are not available and still have to be proven safe and effective before clinical authorization by regulatory institutions. Another disadvantage of the method described in Patent Application No. US20100137982 is the fact that it requires the surgeon to incorporate new surgical steps and maneuvers, such as engagement and interlocking between IOL parts and capsule incision features. These maneuvers can result challenging, difficult to learn and could lead to unexpected complications. The method of the present invention incorporates visible alignment marks in capsule 402 of eye 68 for IOL positioning, rotation and centration, with the advantage that it can be practiced with all currently available toric IOLs.

### SUMMARY OF THE INVENTION

The present invention provides methods and apparatus to precisely seat an IOL within the capsule of an eye of a patient by using a short pulse laser to create a capsular incision with visible marks or features indicative of the preferred rotational axis for implantation of an astigmatic IOL. This can be accomplished by incorporating diametrically opposed features to the enclosed capsule incision. Alternatively, laser marks or incisions can be located peripheral to the main capsular incision as guidance signs for rotational alignment of the astigmatism correcting IOL.

An imaginary straight line traced over the opposing features, marks or incisions is planned to coincide or be parallel to the desired axis of implantation of the astigmatic IOL. Usually, the orientation of this line corresponds to the steep axis of the astigmatism of the patient's eye and with which a line traced over opposing marks on the IOL indicative of the IOL flat axis must coincide. Other conventions for astigmatic IOL alignment can exist. Also, the desired axis of implantation of an astigmatic IOL within the eye can be deliberately shifted from the corneal preoperative steepest axis when using formulas that may account for surgically induced astigmatism or for expected age-induced shifts in corneal astigmatism.

The same axis matching effect can be achieved without the visualization by the surgeon of these imaginary lines, as for example, by seeking direct coincidence of each opposing marks on the capsule and on the lens. The fact that the lens capsule is transparent makes pursuing this objective easy for a surgeon performing standard IOL rotation maneuvers.

A method for inserting an intraocular lens in a patient's eye includes detecting traceable landmarks in the eye of the patient, such as iris and limbal features including blood vessels, pigment marks and variations, detecting the astigmatism of the patients cornea, registering the mayor and minor axis of the corneal astigmatism to the detected eye landmarks, generating a light beam, deflecting the light beam using a scanner to form an enclosed treatment pattern that includes a visible registration feature linkable to the recorded eye landmarks previously registered to the corneal astigmatism mayor and minor axis, delivering the enclosed treatment pattern to target tissue in the patient's eye to form an enclosed incision including the registration feature, and placing an intraocular lens within the enclosed capsular incision, the intraocular lens having intraocular lens astigmatism axis marks that the surgeon aligns with the visible capsular registration feature of the enclosed incision.

Alternatively, a method of inserting an intraocular lens in a patient's eye, comprising detecting traceable eye landmarks, detecting the astigmatism of the cornea, registering the axis of the corneal astigmatism to the traceable eye landmarks, generating a light beam, deflecting the light beam using a scanner to form an enclosed treatment pattern and a registration pattern peripheral to the enclosed treatment pattern which is linkable to the recorded eye landmarks registered to the corneal astigmatism axis and placing an intraocular lens within the enclosed incision, wherein the intraocular lens has a lens astigmatism axis visible registration feature that that the surgeon aligns with the visible registration feature in the form of incisions/marks set peripheral to a main central capsulorhexis incision.

Astigmatism-correcting IOLs need to be placed not only at the correct location within a capsule 402 of the eye 68, but also need to be delivered at the correct rotational/clocking angle. This because these IOLs have inherent optical rotational asymmetries, unlike non-astigmatic IOLs. Precise rotational IOL implantation can also be important for non-astigmatic IOLs. This invention allows for accurate rotational positioning of any IOL that could take advantage of a particular rotational position, as long as the IOL has identifiable rotatory position marks that can be aligned with the laser enclosed incision marks or features.

Not only precise rotational delivery can be important for IOLs such as with toric IOLs. Also IOL centration can be important, particularly for special IOLs such as multifocal IOLs. Accurate IOL centration can be referenced to the optical axis of the eye, to the center of the pupil (photopic or mesopic), or to other landmarks of eye 68. The capsulorhexis incision IOL positioning clues of the present invention can also be used for accurate IOL centration referenced to a selected eye landmark selected by an operator using UI 306 and/or system 890.

Other objects and features of the present invention will become apparent by a review of the specification, claims and appended figures.

### BRIEF DESCRIPTION OF THE DRAWINGS

FIG. 1 is a schematic diagram of the optical beam scanning system.

FIG. 2 is an optical diagram showing an alternative beam combining scheme.

FIG. 3 is a schematic diagram of the optical beam scanning system with an alternative OCT configuration.

FIG. 4 is a schematic diagram of the optical beam scanning system with another alternative OCT combining scheme.

FIG. 5 is a schematic drawing of a diagnostic unit designed to capture astigmatism axis and visible eye features to produce an axis registration file between both ocular features.

FIG. 6A is one model of an astigmatism correcting IOL with visible axis marks.

FIG. 6B is another model of an astigmatism correcting IOL with visible axis marks.

FIG. 6C is a sectorial view of the IOL from FIG. 6B showing the axis indicating features in detail.

FIG. 6D is another view of the IOL from FIG. 6B with an imaginary line traced along the axis indicating visible marks.

FIG. 7A depicts a standard capsulorhexis incision that can be made manually and more accurately using a UF light source and scanner.

FIG. 7B is a preferred embodiment of the present invention with the capsular incision including inward directed visible axis marking features to guide a surgeon for proper astigmatic IOL axis positioning.

FIG. 7C is another view of the capsule incision from FIG. 7B with an imaginary line traced along the axis indicating marking features.

FIG. 8A is another embodiment of the present invention with the capsular incision including outward directed visible axis marking features to guide a surgeon for proper astigmatic IOL axis positioning.

FIG. 8B is another embodiment of the present invention with the capsular incision including a plurality of inward and outward directed visible axis marking features to guide a surgeon for proper astigmatic IOL axis positioning.

FIG. 8C illustrates a plurality of imaginary parallel lines traced over neighbor axis marking features to guide a surgeon for proper astigmatic IOL axis positioning.

FIG. 9A is an schematic diagram of a state of the art clinically available astigmatic correcting IOL

FIG. 9B shows the IOL from FIG. 9A delivered into final position inside a capsule perfectly aligned with the axis guidance marks in the capsule enclosed incision.

FIG. 10 is an exploded image showing the limbal and iris features in an eye together with capsule incision with marks, the astigmatism axis K-map and astigmatic correcting IOL, each having imaginary lines that must be aligned to obtain optimal astigmatism correction.

FIG. 11A shows the IOL from FIG. 9A delivered centered with the capsule incision but not aligned regarding astigmatism axis as the IOL axis marks and the capsule incision marks do not coincide. This lens requires rotation inside the capsule to achieve optimal astigmatism axis matching.

FIG. 11B shows the IOL from FIG. 9A delivered into the capsule not centered with the capsule incision and not aligned regarding astigmatism. This lens requires rotation and centration inside the capsule to achieve optimal astigmatism axis matching and centration

FIG. 12A shows the IOL from FIG. 9A delivered into final position inside a capsule perfectly aligned with the axis guidance marks in the capsule enclosed incision and for further illustration where an IOL axis imaginary line and a capsule incision axis marks imaginary line totally coincide.

FIG. 12B shows the IOL from FIG. 9A delivered inside a capsule perfectly aligned with the axis guidance marks in the capsule enclosed incision but with the optic portion slightly decentered, showing for further illustration that the IOL axis imaginary line and the capsule incision axis marks imaginary line are parallel but not coincident.

FIG. 13A is an embodiment where the astigmatism axis indicating features take the form of small enclosed incisions that lye peripheral to the main capsule incision.

FIG. 13B is an embodiment where the astigmatism axis indicating features are in the form visible marks produced in the capsule tissue by system 2 in the periphery of the main capsule incision.

FIG. 14 is a high magnification photograph captured during real surgery of an eye where the preferred embodiment of the present invention illustrated in FIG. 7B is practiced with advantage. A capsule enclosed incision has an inward directed contour deviation used by the surgeon as a guide to precisely deliver an astigmatism correcting IOL with axis marks.

### DETAILED DESCRIPTION OF THE PREFERRED EMBODIMENTS

The techniques and systems disclosed herein provide many advantages over the current standard of care. Specifically, rapid and precise openings in the lens capsule further including features to aid a surgeon to accurately deliver into final position astigmatism-correcting IOLs are enabled using 3-dimensional patterned laser cutting. In contrast, the controllable, patterned laser techniques described herein may be used to create incisions and/or laser marks in virtually any position in the anterior and/or posterior capsule(s) and in virtually any shape. Furthermore, these capsular incisions and/or marks can be accurately positioned to guide a surgeon to precisely deliver an optically asymmetric IOL that requires to be precisely positioned regarding its rotational orientation.

Moreover, the controllable, patterned laser techniques described herein also have available and/or utilize precise lens capsule size, measurement and other dimensional information that allows the marking and/or the incision or opening formation while minimizing impact on surrounding tissue.

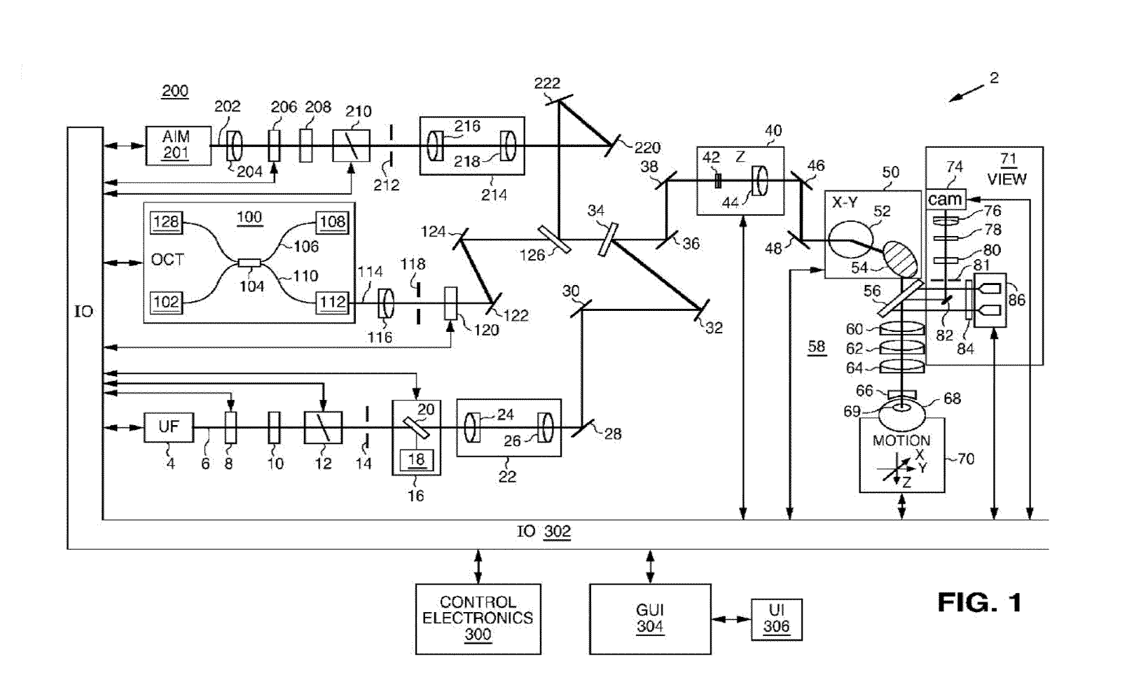

The present invention can be implemented by a system that projects or scans an optical beam into a patient's eye 68, such as system 2 shown in FIG. 1 which includes an ultrafast (UF) light source 4 (e.g. a femtosecond laser). Using this system, a beam may be scanned in a patient's eye in three dimensions: X, Y, Z. In this embodiment, the UF wavelength can vary between 1010 nm to 1100 nm and the pulse width can vary from 100 fs to 10000 fs. The pulse repetition frequency can also vary from 10 kHz to 250 kHz. Safety limits with regard to unintended damage to non-targeted tissue bound the upper limit with regard to repetition rate and pulse energy; while threshold energy, time to complete the procedure and stability bound the lower limit for pulse energy and repetition rate. The peak power of the focused spot in the eye 68 and specifically within the crystalline lens 69 and anterior capsule of the eye is sufficient to produce optical breakdown and initiate a plasma-mediated ablation process. Near-infrared wavelengths are preferred because linear optical absorption and scattering in biological tissue is reduced across that spectral range. As an example, laser 4 may be a repetitively pulsed 1035 nm device that produces 500 fs pulses at a repetition rate of 100 kHz and an individual pulse energy in the ten microjoule range.

The laser 4 is controlled by control electronics 300, via an input and output device 302, to create optical beam 6. Control electronics 300 may be a computer, microcontroller, etc. In this example, the entire system is controlled by the controller 300, and data moved through input/output device IO 302. A graphical user interface GUI 304 may be used to set system operating parameters, process user input (UI) 306 on the GUI 304, and display gathered information such as images of ocular structures.

The generated UF light beam 6 proceeds towards the patient eye 68 passing through half-wave plate, 8, and linear polarizer, 10. The polarization state of the beam can be adjusted so that the desired amount of light passes through half-wave plate 8 and linear polarizer 10, which together act as a variable attenuator for the UF beam 6. Additionally, the orientation of linear polarizer 10 determines the incident polarization state incident upon beamcombiner 34, thereby optimizing beamcombiner throughput.

The UF beam proceeds through a shutter 12, aperture 14, and a pickoff device 16. The system controlled shutter 12 ensures on/off control of the laser for procedural and safety reasons. The aperture sets an outer useful diameter for the laser beam and the pickoff monitors the output of the useful beam. The pickoff device 16 includes of a partially reflecting mirror 20 and a detector 18. Pulse energy, average power, or a combination may be measured using detector 18. The information can be used for feedback to the half-wave plate 8 for attenuation and to verify whether the shutter 12 is open or closed. In addition, the shutter 12 may have position sensors to provide a redundant state detection.

The beam passes through a beam conditioning stage 22, in which beam parameters such as beam diameter, divergence, circularity, and astigmatism can be modified. In this illustrative example, the beam conditioning stage 22 includes a 2 element beam expanding telescope comprised of spherical optics 24 and 26 in order to achieve the intended beam size and collimation. Although not illustrated here, an anamorphic or other optical system can be used to achieve the desired beam parameters. The factors used to determine these beam parameters include the output beam parameters of the laser, the overall magnification of the system, and the desired numerical aperture (NA) at the treatment location. In addition, the optical system 22 can be used to image aperture 14 to a desired location (e.g. the center location between the 2-axis scanning device 50 described below). In this way, the amount of light that makes it through the aperture 14 is assured to make it through the scanning system. Pickoff device 16 is then a reliable measure of the usable light.

After exiting conditioning stage 22, beam 6 reflects off of fold mirrors 28, 30, & 32. These mirrors can be adjustable for alignment purposes. The beam 6 is then incident upon beam combiner 34. Beamcombiner 34 reflects the UF beam 6 (and transmits both the OCT 114 and aim 202 beams described below). For efficient beamcombiner operation, the angle of incidence is preferably kept below 45 degrees and the polarization where possible of the beams is fixed. For the UF beam 6, the orientation of linear polarizer 10 provides fixed polarization.

Following the beam combiner 34, the beam 6 continues onto the z-adjust or Z scan device 40. In this illustrative example the z-adjust includes a Galilean telescope with two lens groups 42 and 44 (each lens group includes one or more lenses). Lens group 42 moves along the z-axis about the collimation position of the telescope. In this way, the focus position of the spot in the patient's eye 68 moves along the z-axis as indicated. In general there is a fixed linear relationship between the motion of lens 42 and the motion of the focus. In this case, the z-adjust telescope has an approximate 2× beam expansion ratio and a 1:1 relationship of the movement of lens 42 to the movement of the focus. Alternatively, lens group 44 could be moved along the z-axis to actuate the z-adjust, and scan. The z-adjust is the z-scan device for treatment in the eye 68. It can be controlled automatically and dynamically by the system and selected to be independent or to interplay with the X-Y scan device described next. Mirrors 36 and 38 can be used for aligning the optical axis with the axis of z-adjust device 40.

After passing through the z-adjust device 40, the beam 6 is directed to the x-y scan device by mirrors 46 & 48. Mirrors 46 & 48 can be adjustable for alignment purposes. X-Y scanning is achieved by the scanning device 50 preferably using two mirrors 52 & 54 under the control of control electronics 300, which rotate in orthogonal directions using motors, galvanometers, or any other well known optic moving device. Mirrors 52 & 54 are located near the telecentric position of the objective lens 58 and contact lens 66 combination described below. Tilting these mirrors 52/54 causes them to deflect beam 6, causing lateral displacements in the plane of UF focus located in the patient's eye 68. Objective lens 58 may be a complex multi-element lens element, as shown, and represented by lenses 60, 62, and 64. The complexity of the lens 58 will be dictated by the scan field size, the focused spot size, the available working distance on both the proximal and distal sides of objective 58, as well as the amount of aberration control. An f-theta lens 58 of focal length 60 mm generating a spot size of 10 um, over a field of 10 mm, with an input beam size of 15 mm diameter is an example. Alternatively, X-Y scanning by scanner 50 may be achieved by using one or more moveable optical elements (e.g. lenses, gratings) which also may be controlled by control electronics 300, via input and output device 302.

The aiming and treatment scan patterns can be automatically generated by the scanner 50 under the control of controller 300. Such patterns may be comprised of a single spot of light, multiple spots of light, a continuous pattern of light, multiple continuous patterns of light, and/or any combination of these. In addition, the aiming pattern (using aim beam 202 described below) need not be identical to the treatment pattern (using light beam 6), but preferably at least defines its boundaries in order to assure that the treatment light is delivered only within the desired target area for patient safety. This may be done, for example, by having the aiming pattern provide an outline of the intended treatment pattern. This way the spatial extent of the treatment pattern may be made known to the user, if not the exact locations of the individual spots themselves, and the scanning thus optimized for speed, efficiency and accuracy. The aiming pattern may also be made to be perceived as blinking in order to further enhance its visibility to the user.

An optional contact lens 66, which can be any suitable ophthalmic lens, can be used to help further focus the optical beam 6 into the patient's eye 68 while helping to stabilize eye position. The positioning and character of optical beam 6 and/or the scan pattern the beam 6 forms on the eye 68 may be further controlled by use of an input device such as a joystick, or any other appropriate user input device (e.g. GUI 304) to position the patient and/or the optical system.

The UF laser 4 and controller 300 can be set to target the surfaces of the targeted structures in the eye 68 and ensure that the beam 6 will be focused where appropriate and not unintentionally damage non-targeted tissue. Imaging modalities and techniques described herein, such as for example, Optical Coherence Tomography (OCT), Purkinje imaging, Scheimpflug imaging, or ultrasound may be used to determine the location and measure the thickness of the lens and lens capsule to provide greater precision to the laser focusing methods, including 2D and 3D patterning. Laser focusing may also be accomplished using one or more methods including direct observation of an aiming beam, Optical Coherence Tomography (OCT), Purkinje imaging, Scheimpflug imaging, ultrasound, or other known ophthalmic or medical imaging modalities and/or combinations thereof. In the embodiment of FIG. 1, an OCT device 100 is described, although other modalities are within the scope of the present invention. An OCT scan of the eye will provide information about the axial location of the anterior and posterior lens capsule, the boundaries of the cataract nucleus, as well as the depth of the anterior chamber. This information is then be loaded into the control electronics 300, and used to program and control the subsequent laser-assisted surgical procedure. The information may also be used to determine a wide variety of parameters related to the procedure such as, for example, the upper and lower axial limits of the focal planes used for cutting the lens capsule and segmentation of the lens cortex and nucleus, and the thickness of the lens capsule among others.

The OCT device 100 in FIG. 1 includes a broadband or a swept light source 102 that is split by a fiber coupler 104 into a reference arm 106 and a sample arm 110. The reference arm 106 includes a module 108 containing a reference reflection along with suitable dispersion and path length compensation. The sample arm 110 of the OCT device 100 has an output connector 112 that serves as an interface to the rest of the UF laser system. The return signals from both the reference and sample arms 106, 110 are then directed by coupler 104 to a detection device 128, which employs either time domain, frequency or single point detection techniques. In FIG. 1, a frequency domain technique is used with an OCT wavelength of 920 nm and bandwidth of 100 nm.

Exiting connector 112, the OCT beam 114 is collimated using lens 116. The size of the collimated beam 114 is determined by the focal length of lens 116. The size of the beam 114 is dictated by the desired NA at the focus in the eye and the magnification of the beam train leading to the eye 68. Generally, OCT beam 114 does not require as high an NA as the UF beam 6 in the focal plane and therefore the OCT beam 114 is smaller in diameter than the UF beam 6 at the beamcombiner 34 location. Following collimating lens 116 is aperture 118 which further modifies the resultant NA of the OCT beam 114 at the eye. The diameter of aperture 118 is chosen to optimize OCT light incident on the target tissue and the strength of the return signal. Polarization control element 120, which may be active or dynamic, is used to compensate for polarization state changes which may be induced by individual differences in corneal birefringence, for example. Mirrors 122 & 124 are then used to direct the OCT beam 114 towards beamcombiners 126 & 34. Mirrors 122 & 124 may be adjustable for alignment purposes and in particular for overlaying of OCT beam 114 to UF beam 6 subsequent to beamcombiner 34. Similarly, beamcombiner 126 is used to combine the OCT beam 114 with the aim beam 202 described below.

Once combined with the UF beam 6 subsequent to beamcombiner 34, OCT beam 114 follows the same path as UF beam 6 through the rest of the system. In this way, OCT beam 114 is indicative of the location of UF beam 6. OCT beam 114 passes through the z-scan 40 and x-y scan 50 devices then the objective lens 58, contact lens 66 and on into the eye 68. Reflections and scatter off of structures within the eye provide return beams that retrace back through the optical system, into connector 112, through coupler 104, and to OCT detector 128. These return back reflections provide the OCT signals that are in turn interpreted by the system as to the location in X, Y Z of UF beam 6 focal location.

OCT device 100 works on the principle of measuring differences in optical path length between its reference and sample arms. Therefore, passing the OCT through z-adjust 40 does not extend the z-range of OCT system 100 because the optical path length does not change as a function of movement of 42. OCT system 100 has an inherent z-range that is related to the detection scheme, and in the case of frequency domain detection it is specifically related to the spectrometer and the location of the reference arm 106. In the case of OCT system 100 used in FIG. 1, the z-range is approximately 1-2 mm in an aqueous environment. Extending this range to at least 4 mm involves the adjustment of the path length of the reference arm within OCT system 100. Passing the OCT beam 114 in the sample arm through the z-scan of z-adjust 40 allows for optimization of the OCT signal strength. This is accomplished by focusing the OCT beam 114 onto the targeted structure while accommodating the extended optical path length by commensurately increasing the path within the reference arm 106 of OCT system 100.

Because of the fundamental differences in the OCT measurement with respect to the UF focus device due to influences such as immersion index, refraction, and aberration, both chromatic and monochromatic, care must be taken in analyzing the OCT signal with respect to the UF beam focal location. A calibration or registration procedure as a function of X, Y Z should be conducted in order to match the OCT signal information to the UF focus location and also to the relate to absolute dimensional quantities.

Observation of an aim beam may also be used to assist the user to directing the UF laser focus. Additionally, an aim beam visible to the unaided eye in lieu of the infrared OCT and UF beams can be helpful with alignment provided the aim beam accurately represents the infrared beam parameters. An aim subsystem 200 is employed in the configuration shown in FIG. 1. The aim beam 202 is generated by an aim beam light source 201, such as a helium-neon laser operating at a wavelength of 633 nm. Alternatively a laser diode in the 630-650 nm range could be used. The advantage of using the helium neon 633 nm beam is its long coherence length, which would enable the use of the aim path as a laser unequal path interferometer (LUPI) to measure the optical quality of the beam train, for example.

Once the aim beam light source generates aim beam 202, the aim beam 202 is collimated using lens 204. The size of the collimated beam is determined by the focal length of lens 204. The size of the aim beam 202 is dictated by the desired NA at the focus in the eye and the magnification of the beam train leading to the eye 68. Generally, aim beam 202 should have close to the same NA as UF beam 6 in the focal plane and therefore aim beam 202 is of similar diameter to the UF beam at the beamcombiner 34 location. Because the aim beam is meant to stand-in for the UF beam 6 during system alignment to the target tissue of the eye, much of the aim path mimics the UF path as described previously. The aim beam 202 proceeds through a half-wave plate 206 and linear polarizer 208. The polarization state of the aim beam 202 can be adjusted so that the desired amount of light passes through polarizer 208. Elements 206 & 208 therefore act as a variable attenuator for the aim beam 202. Additionally, the orientation of polarizer 208 determines the incident polarization state incident upon beamcombiners 126 and 34, thereby fixing the polarization state and allowing for optimization of the beamcombiners' throughput. Of course, if a semiconductor laser is used as aim beam light source 200, the drive current can be varied to adjust the optical power.

The aim beam 202 proceeds through a shutter 210 and aperture 212. The system controlled shutter 210 provides on/off control of the aim beam 202. The aperture 212 sets an outer useful diameter for the aim beam 202 and can be adjusted appropriately. A calibration procedure measuring the output of the aim beam 202 at the eye can be used to set the attenuation of aim beam 202 via control of polarizer 206.

The aim beam 202 next passes through a beam conditioning device 214. Beam parameters such as beam diameter, divergence, circularity, and astigmatism can be modified using one or more well known beam conditioning optical elements. In the case of an aim beam 202 emerging from an optical fiber, the beam conditioning device 214 can simply include a beam expanding telescope with two optical elements 216 and 218 in order to achieve the intended beam size and collimation. The final factors used to determine the aim beam parameters such as degree of collimation are dictated by what is necessary to match the UF beam 6 and aim beam 202 at the location of the eye 68. Chromatic differences can be taken into account by appropriate adjustments of beam conditioning device 214. In addition, the optical system 214 is used to image aperture 212 to a desired location such as a conjugate location of aperture 14.

The aim beam 202 next reflects off of fold mirrors 222 & 220, which are preferably adjustable for alignment registration to UF beam 6 subsequent to beam combiner 34. The aim beam 202 is then incident upon beam combiner 126 where the aim beam 202 is combined with OCT beam 114. Beamcombiner 126 reflects the aim beam 202 and transmits the OCT beam 114, which allows for efficient operation of the beamcombining functions at both wavelength ranges. Alternatively, the transmitting and reflect functions of beamcombiner 126 can be reversed and the configuration inverted. Subsequent to beamcombiner 126, aim beam 202 along with OCT beam 114 is combined with UF beam 6 by beamcombiner 34.

A device for imaging the target tissue on or within the eye 68 is shown schematically in FIG. 1 as imaging system 71. Imaging system includes a camera 74 and an illumination light source 86 for creating an image of the target tissue. The imaging system 71 gathers images which may be used by the system controller 300 for providing pattern centering about or within a predefined structure. The illumination light source 86 for the viewing is generally broadband and incoherent. For example, light source 86 can include multiple LEDs as shown. The wavelength of the viewing light source 86 is preferably in the range of 700 nm to 750 nm, but can be anything which is accommodated by the beamcombiner 56, which combines the viewing light with the beam path for UF beam 6 and aim beam 202 (beamcombiner 56 reflects the viewing wavelengths while transmitting the OCT and UF wavelengths). The beamcombiner 56 may partially transmit the aim wavelength so that the aim beam 202 can be visible to the viewing camera 74. Optional polarization element 84 in front of light source 86 can be a linear polarizer, a quarter wave plate, a half-wave plate or any combination, and is used to optimize signal. A false color image as generated by the near infrared wavelength is acceptable.

The illumination light from light source 86 is directed down towards the eye using the same objective lens 58 and contact lens 66 as the UF and aim beam 6, 202. The light reflected and scattered off of various structures in the eye 68 are collected by the same lenses 58 & 66 and directed back towards beamcombiner 56. There, the return light is directed back into the viewing path via beam combiner and mirror 82, and on to camera 74. Camera 74 can be, for example but not limited to, any silicon based detector array of the appropriately sized format. Video lens 76 forms an image onto the camera's detector array while optical elements 80 & 78 provide polarization control and wavelength filtering respectively. Aperture or iris 81 provides control of imaging NA and therefore depth of focus and depth of field. A small aperture provides the advantage of large depth of field which aids in the patient docking procedure. Alternatively, the illumination and camera paths can be switched. Furthermore, aim light source 200 can be made to emit in the infrared which would not directly visible, but could be captured and displayed using imaging system 71.

Coarse adjust registration is usually needed so that when the contact lens 66 comes into contact with the cornea, the targeted structures are in the capture range of the X, Y scan of the system. Therefore a docking procedure is preferred, which preferably takes in account patient motion as the system approaches the contact condition (i.e. contact between the patient's eye 68 and the contact lens 66. The viewing system 71 is configured so that the depth of focus is large enough such that the patient's eye 68 and other salient features may be seen before the contact lens 66 makes contact with eye 68.

Preferably, a motion control system 70 is integrated into the overall control system 2, and may move the patient, the system 2 or elements thereof, or both, to achieve accurate and reliable contact between contact lens 66 and eye 68. Furthermore, a vacuum suction subsystem and flange may be incorporated into system 2, and used to stabilize eye 68. The alignment of eye 68 to system 2 via contact lens 66 may be accomplished while monitoring the output of imaging system 71, and performed manually or automatically by analyzing the images produced by imaging system 71 electronically by means of control electronics 300 via IO 302. Force and/or pressure sensor feedback may also be used to discern contact, as well as to initiate the vacuum subsystem.

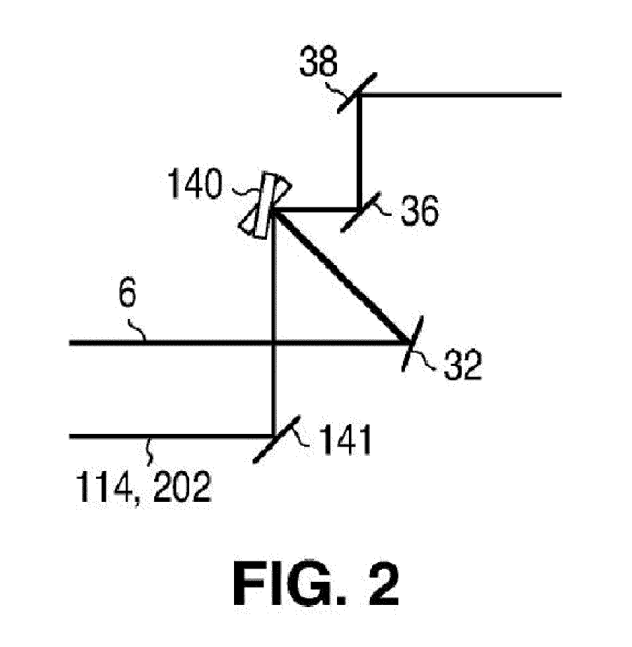

An alternative beam-combining configuration is shown in the alternate embodiment of FIG. 2. For example, the passive beamcombiner 34 in FIG. 1 can be replaced with an active combiner 140 in FIG. 2. The active beamcombiner 34 can be a moving or dynamically controlled element such as a galvanometric scanning mirror, as shown. Active combiner 140 changes it angular orientation in order to direct either the UF beam 6 or the combined aim and OCT beams 202, 114 towards the scanner 50 and eventually eye 68 one at a time. The advantage of the active combining technique is that it avoids the difficulty of combining beams with similar wavelength ranges or polarization states using a passive beam combiner. This ability is traded off against the ability to have simultaneous beams in time and potentially less accuracy and precision due to positional tolerances of active beam combiner 140.

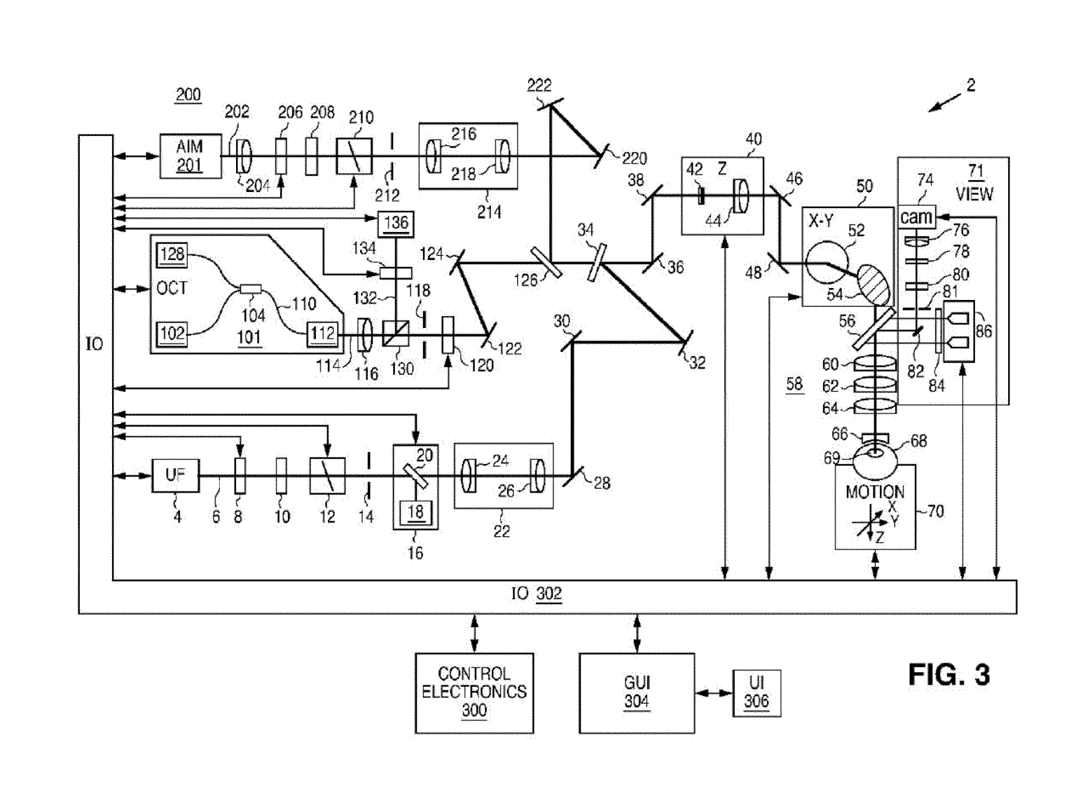

Another alternate embodiment is shown in FIG. 3 which is similar to that of FIG. 1 but utilizes an alternate approach to OCT 100. In FIG. 3, OCT 101 is the same as OCT 100 in FIG. 1, except that the reference arm 106 has been replaced by reference arm 132. This free-space OCT reference arm 132 is realized by including beamsplitter 130 after lens 116. The reference beam 132 then proceeds through polarization controlling element 134 and then onto the reference return module 136. The reference return module 136 contains the appropriate dispersion and path length adjusting and compensating elements and generates an appropriate reference signal for interference with the sample signal. The sample arm of OCT 101 now originates subsequent to beamsplitter 130. The potential advantages of this free space configuration include separate polarization control and maintenance of the reference and sample arms. The fiber based beam splitter 104 of OCT 101 can also be replaced by a fiber based circulator. Alternately, both OCT detector 128 and beamsplitter 130 might be moved together as opposed to reference arm 136.

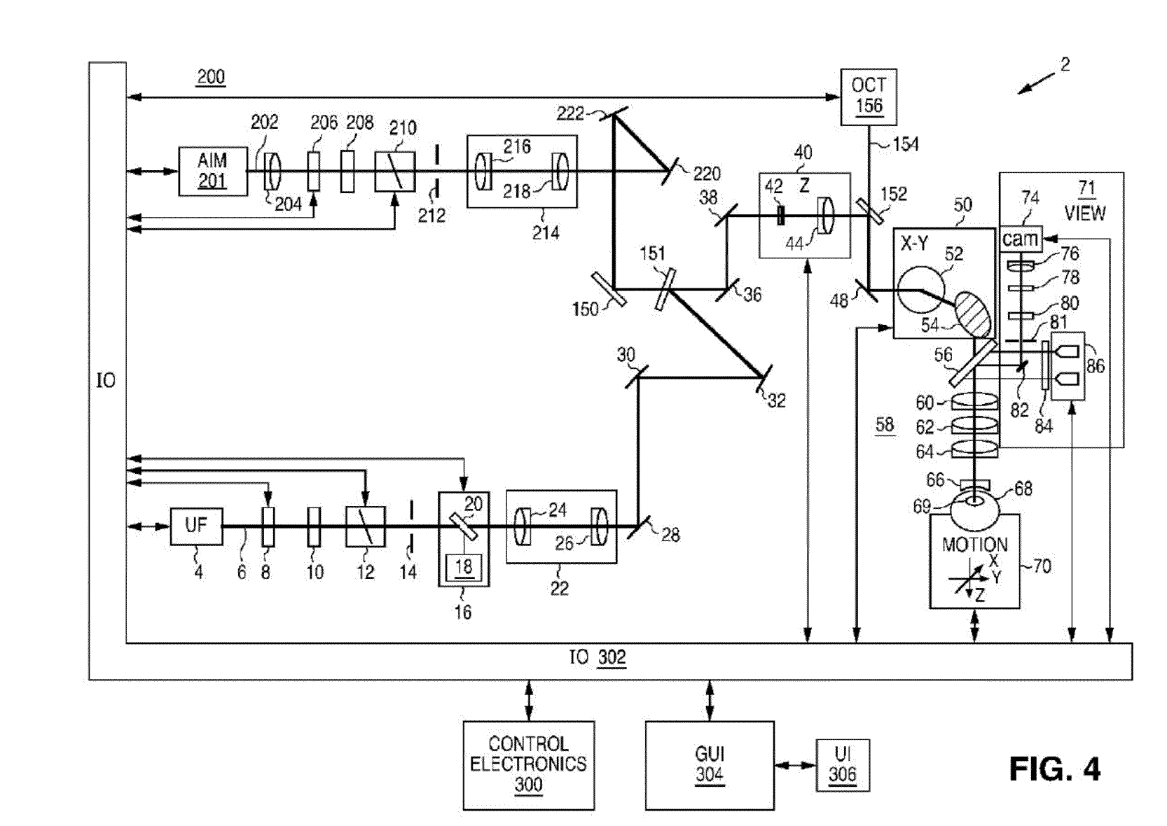

FIG. 4 shows another alternative embodiment for combining OCT beam 114 and UF beam 6. In FIG. 4, OCT 156 (which can include either of the configurations of OCT 100 or 101) is configured such that its OCT beam 154 is coupled to UF beam 6 after the z-scan 40 using beamcombiner 152. In this way, OCT beam 154 avoids using the z-adjust. This allows the OCT 156 to possibly be folded into the beam more easily and shortening the path length for more stable operation. This OCT configuration is at the expense of an optimized signal return strength as discussed with respect to FIG. 1. There are many possibilities for the configuration of the OCT interferometer, including time and frequency domain approaches, single and dual beam methods, swept source, etc, as described in U.S. Pat. Nos. 5,748,898; 5,748,352; 5,459,570; 6,111,645; and 6,053,613 (which are incorporated herein by reference.)

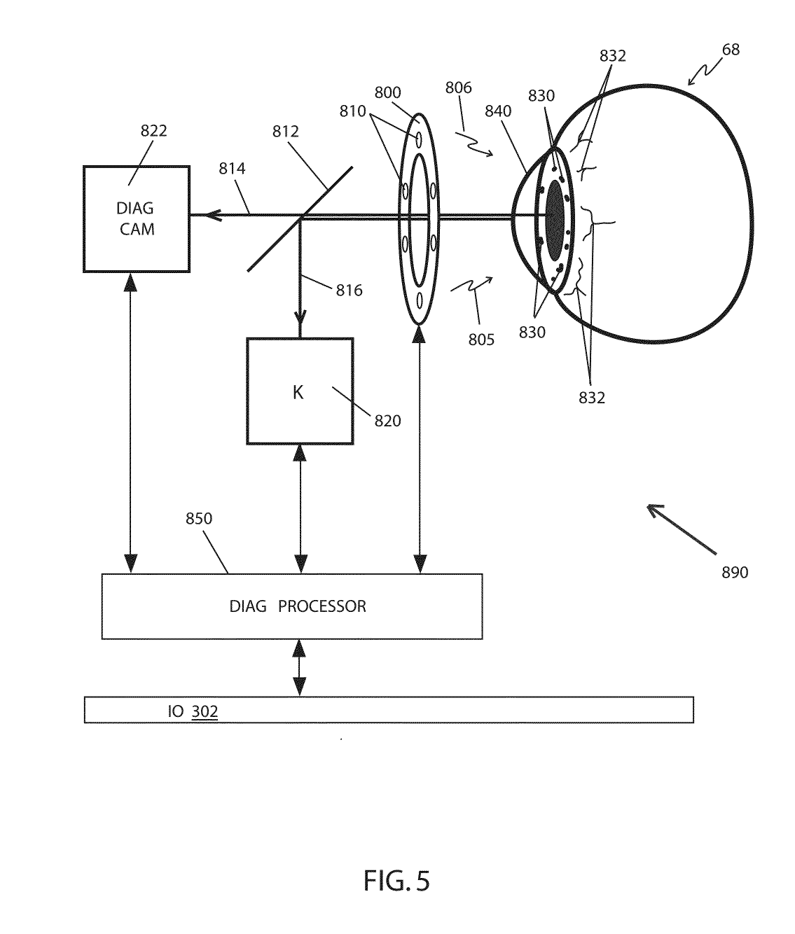

FIG. 5 is an exemplary schematic illustration of a system 890 to simultaneously capture the curvatures of a cornea 840 including corneal astigmatism flat and steep axis, as well as traceable features from the eye 68 and iris surface. These features can be for example the pattern of blood vessels 832, color shifts and pigment marks and features of the iris 830, as well as other eye features such as retinal vessels, etc. System 890 provides a diffuse illumination system 800 and a patterned illumination system 810. Diffuse illumination system 800 producing beam 805 is used to acquire an image by camera 822 from optical path 814 traversing a partial mirror 812. This image includes the traceable features 832 and 830 of eye 68. Patterned illumination is used to capture a reflected pattern image 816 from the cornea by keratometry sensor 820 after reflection by partial mirror 812. This pattern image can be computed to an astigmatism magnitude and axis. The functions described for detectors 820 and 822 can be integrated into the same detector device. Also, other diagnostic methods such as OCT can be used for the same purpose of determining eye features and keratometry values and axis. A processor 850 in system 890 generates an output file including the traceable features of the eye registered with at least the steep axis of astigmatism of the eye's cornea. Astigmatic axis detection with image capture and registration can be performed well before the process of performing the capsule incision using system 2, using standard methods for file transfer into system 2.

Alternatively, system 2 can incorporate a sub-system module performing as system 890, usually prior to eye docking for laser capsular incision. Anyway, these traceable features and astigmatism axis registered to them are provided to system 2 through I/O interface 302. Other eye features such as pupil diameter and pupil centration under varying illumination conditions can be recorder using system 890 and used for practicing the method of the present invention.

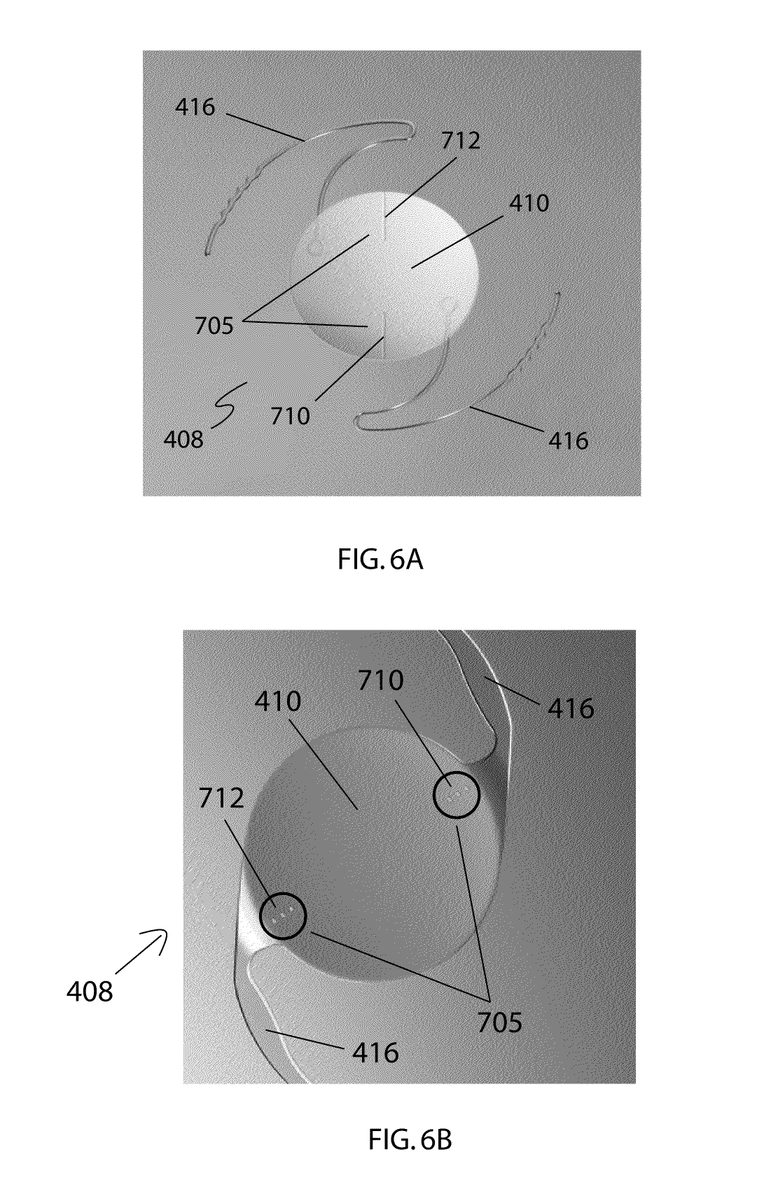

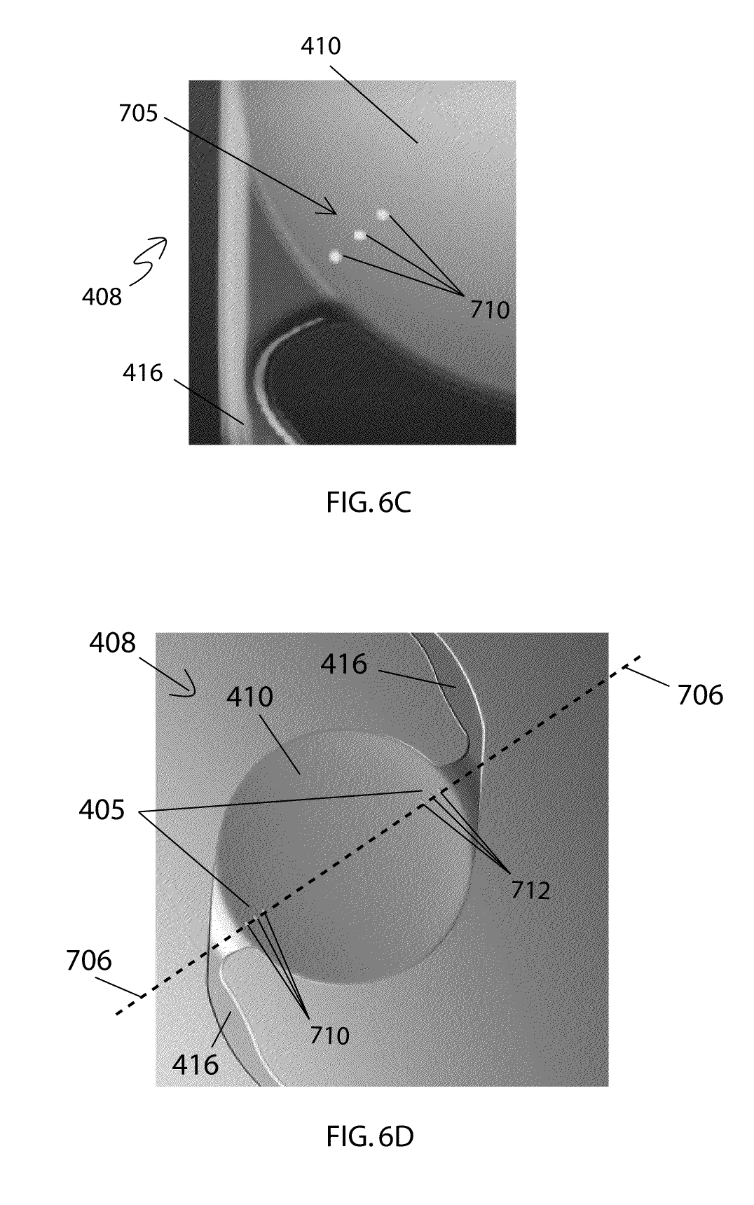

Current state-of-the-art astigmatism correcting IOLs are manufactured incorporating accurate axis marks visible to a surgeon during lens implantation. FIG. 6A and FIG. 6B depict examples of two currently available astigmatism correcting IOLs. An intraocular lens 408 is typically composed of an optic portion 410 and a haptics portion 416. Toric IOL astigmatism axis alignment marks 705 can be etched into the materials of their host elements, or alternately imprinted upon them. Another property of these rotationally critical IOLs is that haptics 416 are designed to prevent any further rotation after a surgeon has completed the IOL implantation maneuvers. In the toric IOL depicted in FIG. 6A, the astigmatism alignment marks consist in lines 710 and 712 radially disposed from the center of the optic portion 410. In FIG. 6B is shown another model of astigmatism correcting IOL with an opposing pair of axis marks 705 (Acrysof Toric IOL, Alcon, USA). Each of these marks is composed by an opposing group of three dots 710 and 712, diametrically disposed across the center of optic portion 410, with all 6 dots fitting into a single straight imaginary line 706 as shown in FIG. 6D. A convention has been adopted with astigmatic IOLs that defines that astigmatism axis marks fall within a line that is parallel to the IOL astigmatic flat axis, this is, the axis that should be aligned to match the steep axis of the astigmatism of a patient to cancel out the corneal astigmatism of the eye 68. A detail from FIG. 6B is depicted in FIG. 6C showing optic portion 410 and haptic portion 416 from toric IOL 408. Astigmatism axis alignment marks 705 consist in a group of three circular marks 710 disposed in a linear array.

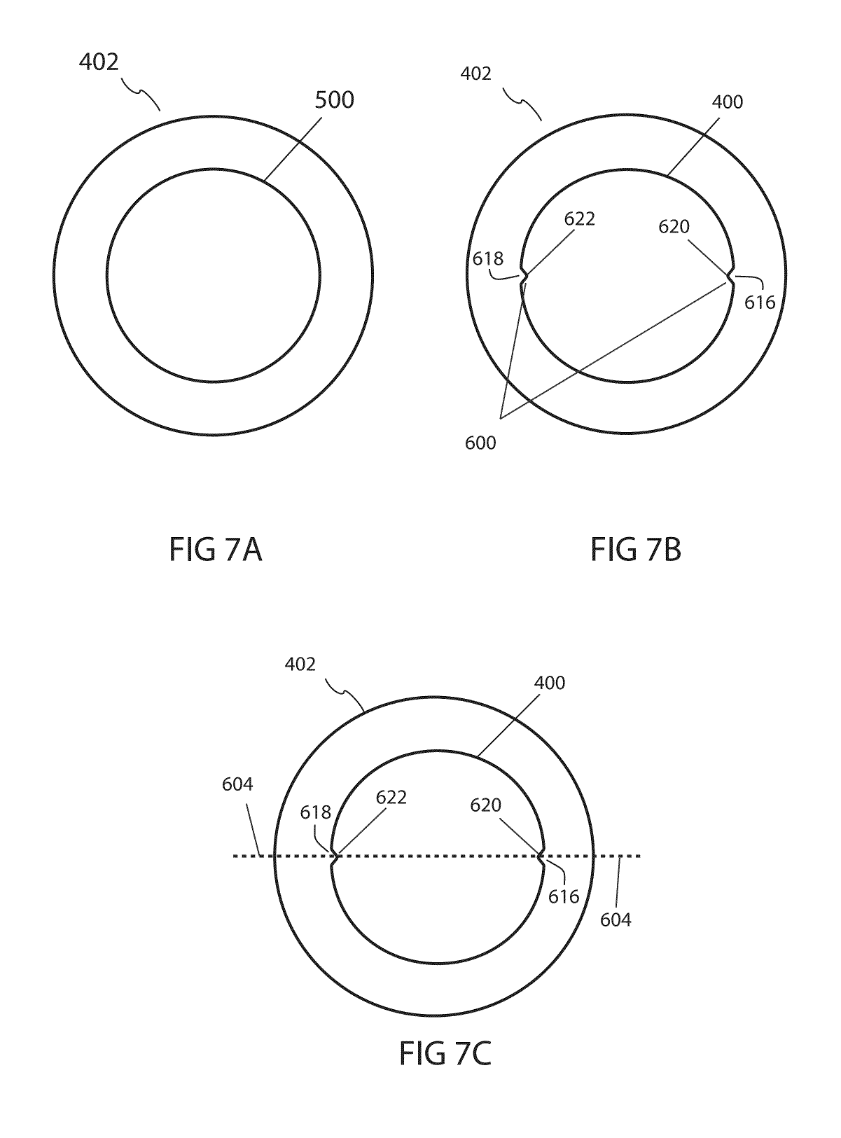

In FIG. 7A is shown a capsulorhexis incision of the prior art 500 within a capsule 402 of the eye 68, carrying no precise clue for the operating surgeon regarding IOL implantation indications such as the axis of a corneal astigmatism of the subject's eye. In FIG. 7B and FIG. 7C is depicted a preferred embodiment of the present invention, which can be implemented using the scanning system 2 and detector system 890 described above. A capsulorhexis incision 400 of the present invention, within a capsule 402 of the eye 68 (which may be created using system 2) incorporates IOL positioning clues such as astigmatism axis identification features 600 that allow precise identification by a surgeon and subsequently accurate positioning of, for example, an astigmatism-correcting intraocular lens (Toric IOL or TIOL) with visible astigmatism axis indication marks 705, 710,712. Incision 400 shown in this example is mainly circular, however, other shapes of main capsulorhexis incisions are possible. Incision 400 may be made continuously, or piecewise to largely maintain the structural integrity of the lens-capsule apparatus of the patient's eye 68. Such incomplete incisions 400 may be thought of as perforated incisions, and may be made to be removed gently in order to minimize their potential to inadvertently extend the capsulorhexis. Either way, incision 400 is an enclosed incision, which for the purposes of this disclosure means that it starts and ends at the same location and encircles a certain amount of tissue therein. The simplest example of an enclosed incision is a circular incision, where a round piece of tissue is encircled by the incision. It follows therefore that an enclosed treatment pattern (i.e. generated by system 2 for forming an enclosed incision) is one that also starts and ends at the same location and defines a space encircled thereby. As seen in FIG. 7B, one key feature of the enclosed incision 400 of the present invention is that it includes an IOL axis orientation feature 600 to aid a surgeon to rotationally align an IOL that incorporates embedded IOL axis marks 705,710,712. For the illustrated circular incision 400 in FIG. 7B, the registration feature 600 consists in a pair of diametrically opposed small curvilinear centripetal flaps 620 and 622, which in combination allow for the accurate placement of an IOL by virtue of the IOL axis indicating marks 710 and 712. Any shape is allowed to these axis indicating flaps as long as the enclosed nature of the incision is preserved. This condition is met when no portions of the incision can naturally extend to the periphery. Even if the axis indicating flaps are terminated centrally in a sharp angle, the enclosed nature of the incision is preserved. Dimensions of capsule axis features can be tailored to IOL axis marks. As seen in FIG. 7C, an imaginary straight line 604 can be traced connecting the apex 620 and 622 of flaps 616 and 618 respectively, in this example at an angle of 0 degrees, indicating that the steep axis of corneal astigmatism of the eye is at 0 degrees. More than alignment with a specific rotational axis, what processor 300 of scanning surgical laser system 2 actually does is to identify the particular registration iris and/or limbal marks of the eye to be operated after docking. Processor 300 also identifies the rotational orientation of the steep axis of the corneal astigmatism of the same eye and displays it through GUI 304. Assignment of an axis value, such as 0 degrees for the present example is for reference for the surgeon only, as the eye can rotate around its Z axis before, and during initial docking. System 2 will locate the limbal and/or iris reference features and will use these to create the capsule incision features 600 aligned with the steep axis of the astigmatism axis registered to the limbal and/or iris features. In this way, rotation of the eye around the Z axis is irrelevant and does not compromise delivery of an accurate astigmatic axis indicating feature while system 2 is creating capsular incision 400. The axis marking feature 600 preserves de enclosed incision nature of capsular incision 400, following a pattern of smooth curves that prevents increased risk of capsule tears. In fact, capsule resistance to expansion is preserved. After completing the docking process for UF light treatment with current systems completely prevent eye movements including rotation in the Z axis. Anyway, if rotation could be an issue, torsional tracking systems exist that can keep track of the movements of the eye during treatment and correct the incision delivery “on the fly” to maintain registration of the axis of capsule features to ocular limbal and/or iris landmarks.

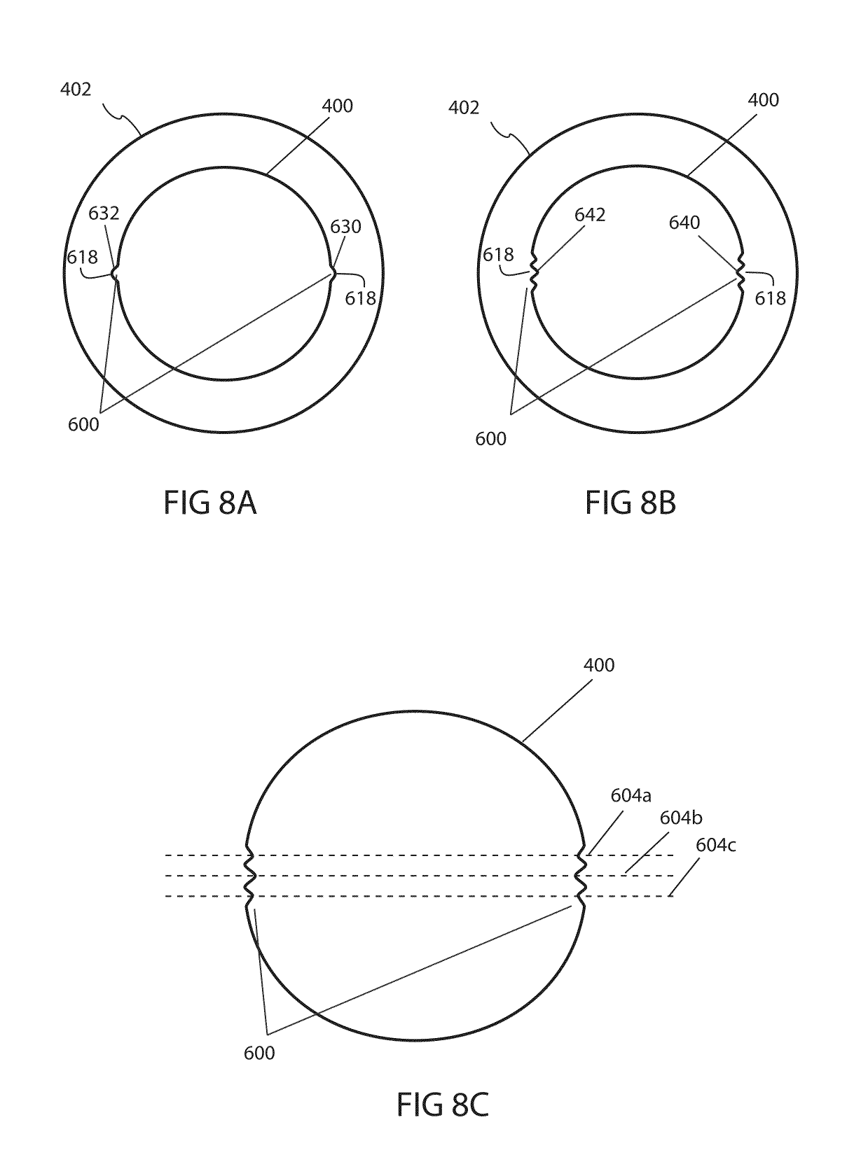

FIG. 8A depicts an alternative embodiment of the present invention where the axis orientation feature 600 shape is reversed, in a way that instead of forming a centripetal flap pair, it is now an outward curvilinear sectorial deviation from the circular contour of the main capsular incision 400. For the illustrated circular incision 400 in FIG. 8A, the registration feature 600 consists in a pair of diametrically opposed small curvilinear outward notches 630 and 632 with apex 616 and 618, which in combination allow for the accurate rotational placement of an IOL by virtue of the IOL axis indicating marks 710 and 712 that should allow the surgeon to seek parallelism of lines 604 and 706 as previously detailed for the preferred embodiment. In FIG. 8B another alternative embodiment is described in which capsule feature 600 corresponds to a pair of diametrically opposed complex contour deviations 640 and 642 included in the main circular shape of capsule incision 400. These features 640 and 642 correspond with each other in a mirror-image fashion permitting to draw a plurality of imaginary parallel lines 604a, 604b and 604c in the present example. As shown in FIG. 8C, with this embodiment, a plurality of imaginary parallel lines drawn across opposite facing peaks and valleys of the multi-curved contour all coincide with the desired axis of implantation of a toric IOL. In this way, toric IOLs that fail to center well with a single pair of axis orientation features can be accurately aligned. System 2 can be programmed to adjust the number of peaks and valleys, their size and the distance between them, always having in mind to maintain a safety profile regarding enclosed incision resistance to tearing and rupture.

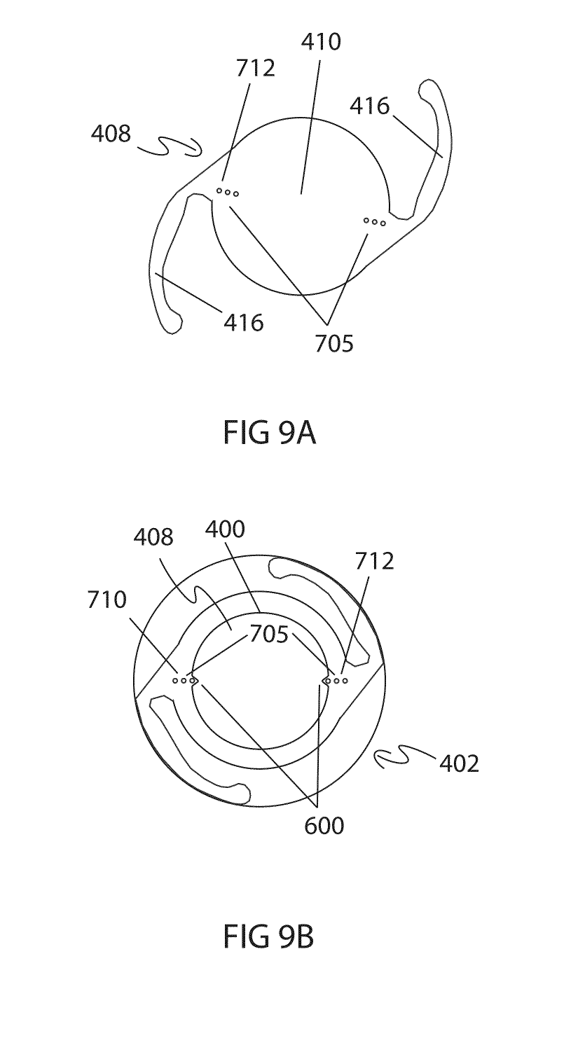

In FIG. 9A is shown a toric IOL 408, with optic portion 410 and positioning haptics 416. Astigmatism axis features 705 showing the IOL flat cylinder axis consist in a pair of diametrically opposed marks 710 and 712 intersecting a straight imaginary line 706. The IOL from FIG. 9A is shown in FIG. 9B inside capsule 402. IOL 408 has IOL astigmatism axis marks 710 and 715 properly aligned with capsule incision IOL axis features 600.

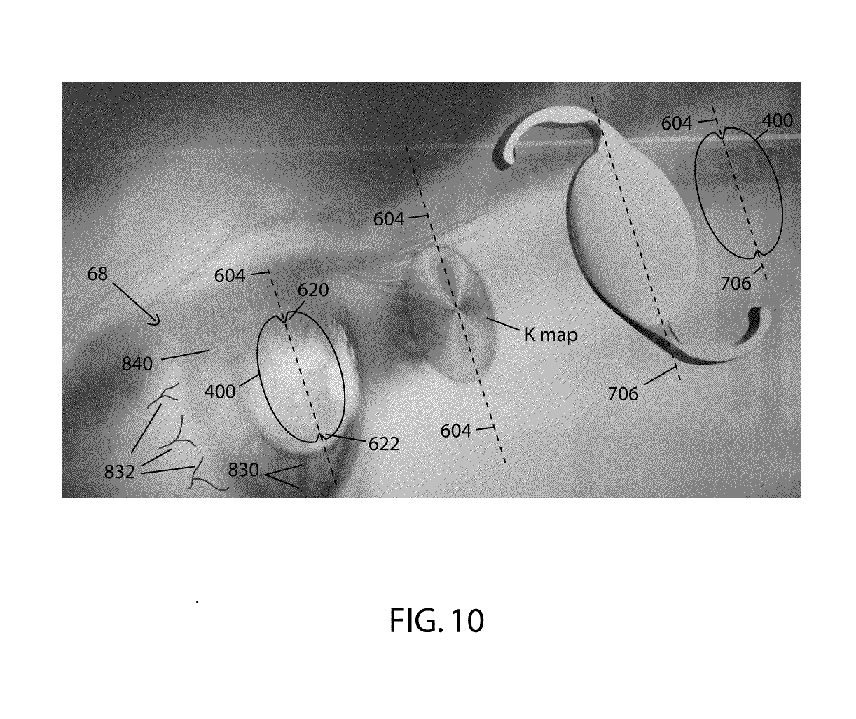

Shown in FIG. 10 is an exploded view of the cornea with dotted line 604 indicating the astigmatism steep axis as illustrated by the topography overlay image in the center, and the desired rotational orientation of a toric IOL with flat IOL astigmatism axis marks aligned with dotted line 706. As illustrated, proper IOL orientation includes achieving parallelism of lines 604 and 706. This is easily achieved following the incision 400 axis features 600 such as marks 620 and 622 that allow imaginary line 604 determination to make parallel with corresponding IOL axis line 706.

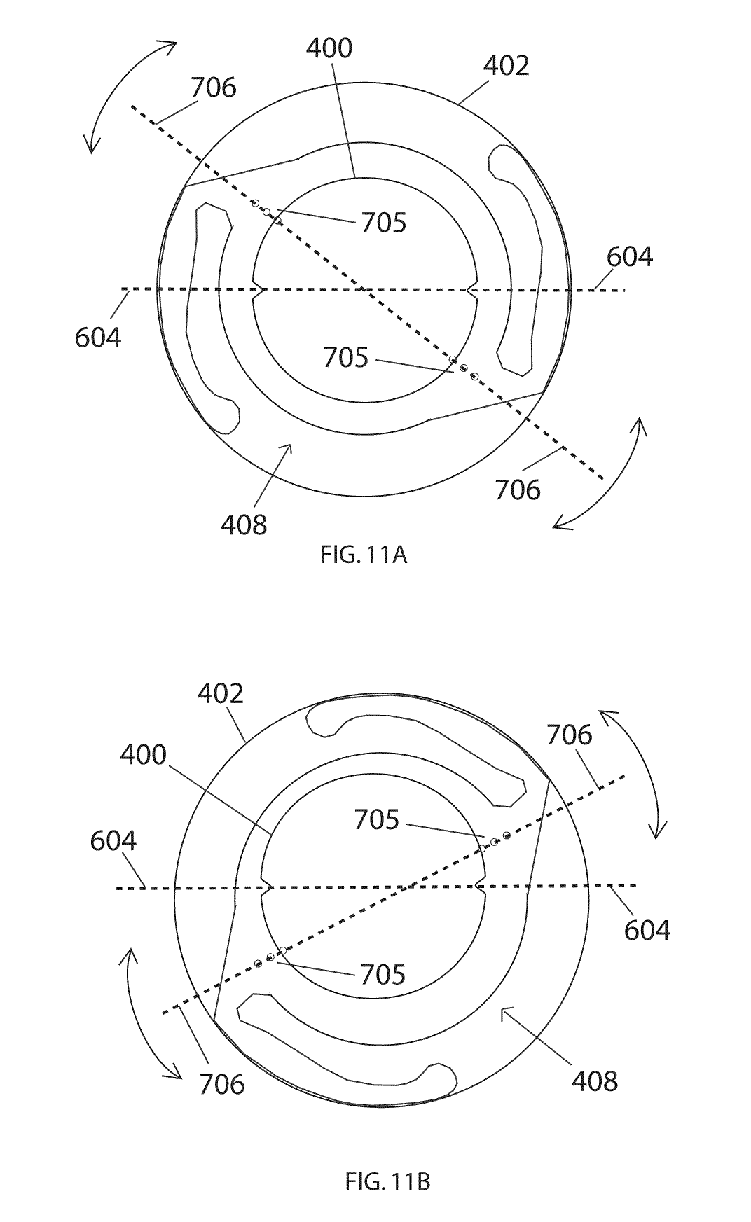

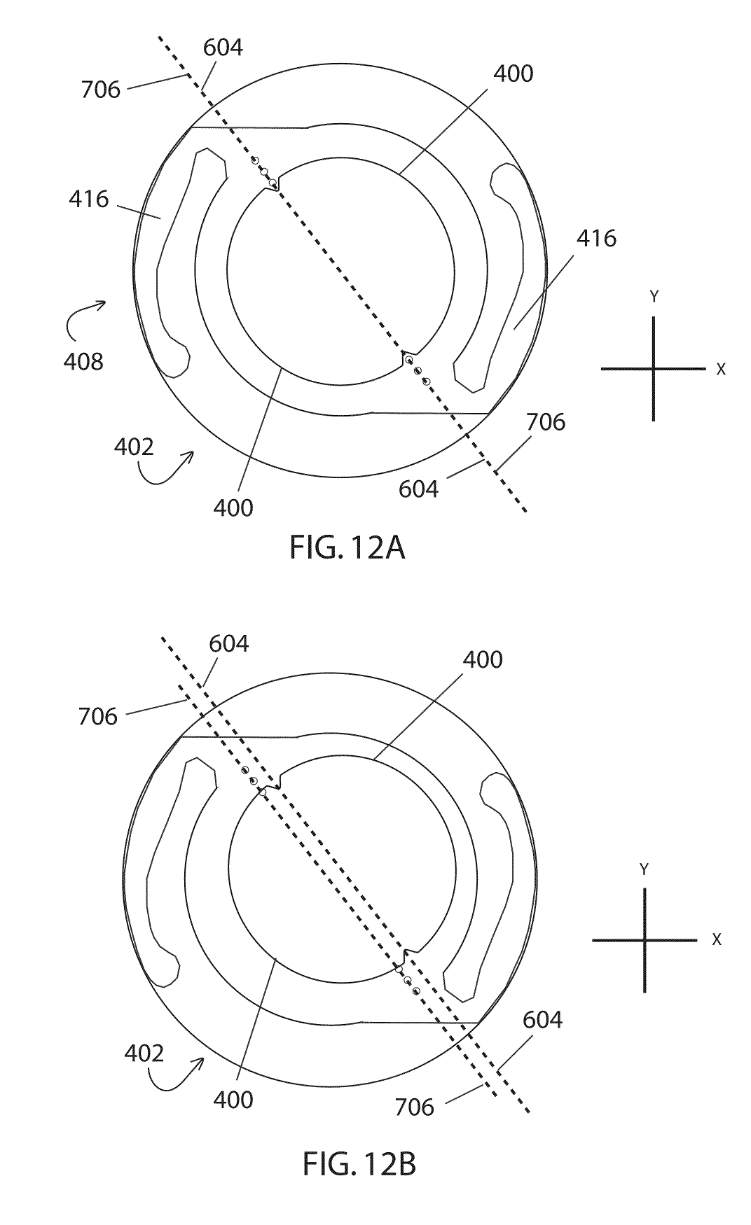

In FIG. 11A and FIG. 11B can be seen inside capsule 402 an IOL 408 with rotational flat axis indication marks usable with the present invention. Enclosed incision 400 has IOL position indication features 600 fitting within imaginary line 604. IOL 408 flat axis indication marks 705 fit within imaginary line 706. In FIG. 11A, the optical portion 410 of lens 408 is well centered with regard to enclosed incision 400. However the lens flat axis defining imaginary line 706 is at a finite angle about +45 degrees clockwise with regard to capsule incision rotary alignment imaginary line 604. In this configuration the flat axis of IOL 408 and the steep axis of eye 68 cornea are not aligned leading to the induction of an unwanted sphere and cylindrical optical value. In FIG. 11B, another misaligned IOL 408 is shown, this time with flat axis line 706 falling about −30 degrees from capsule incision axis line 604. In this case the optical portion 410 is also decentered from the geometric center of capsule incision 400. An operating surgeon using the present invention for advantage can easily align line 706 with line 604 using standard surgical maneuver to rotate IOL 408 into position. For maximum cylinder correction accuracy, line 706 must be parallel to line 604. When opposed axis features 600 are diametrically located regarding incision 400 crossing the center of optical portion 410 these marks are also helpful for accurate centration of IOL 408. When lines 706 and 604 are not only parallel, indicating an accurate axis match, but also they coincide, overlaying one on top of each other, there is both an axis match and an accurate centration. A surgeon should focus on delivering the IOL with lines 706 and 604 parallel and as close as possible between each other maximizing IOL rotary axis matching and optic centration accuracy inside capsule 402. FIG. 12A is an illustration of the desirable end position of IOL 408 inside capsule 402, with lines 604 and 706 in maximum rotary and X-Y coincidence. Asymmetries of the equatorial region of capsule 402 and/or de-centration of enclosed incision 400 can make IOL 408 unable to remain centered stable in the optimal position depicted in FIG. 12A. In this situation shown in FIG. 12B, the surgeon must maneuver to deliver IOL 408 with IOL line 706 and capsule line 604, as parallel as possible and second, as near as possible between them. Some elaborate axis features 600 as shown in FIG. 8C are helpful for a surgeon to obtain parallelism between lines 604 and 706 even when the lens is decentered by using the multiple parallel imaginary lines 604a, 604b and 604c shown in this example. These lines can also give an accurate estimation of the magnitude of de-centration when calibrated to known magnitudes.

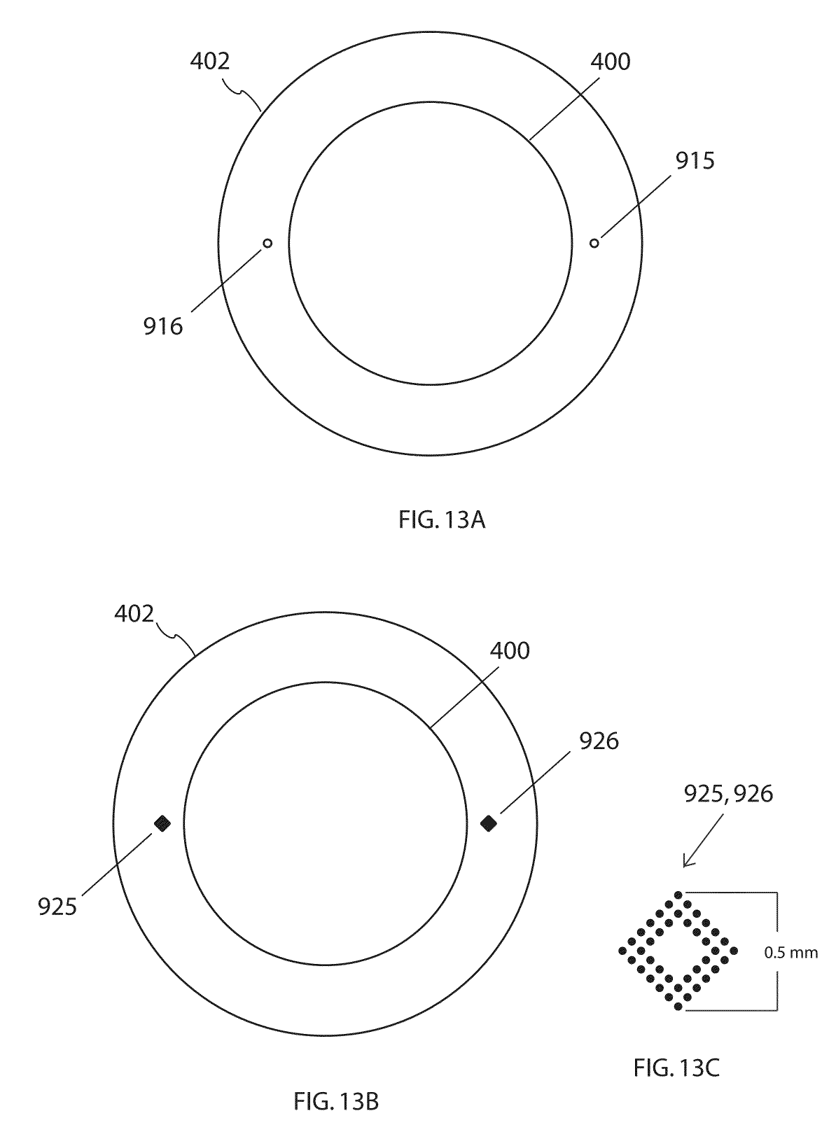

Shown in FIG. 13A and FIG. 13B is an alternative embodiment in which the alignment features no longer consist in feature deformations or alterations of the contour of capsule incision 400, but instead, to visible marks or incisions made by scanning system 2 in an area of capsule 402 outside the boundaries of the main capsule incision 400 constructed to admit and retain the IOL.

Shown in FIG. 13A is an embodiment, as a mode of example only, where diametrically opposed axis marks 915 and 916 lying in capsule 402 consist in a pair of enclosed incisions serving the purpose of being visible marks for a surgeon to trace an imaginary line 604 to accurately rotationally deliver a toric IOL 408. These enclosed incision marks can be made with reduced energy, with reduced spots, and/or with increased spacing of the laser treatment spots in a way that the center tissue can be retained in position. Such incomplete incisions 400 may be thought of as perforated incisions. The enclosed incisions pattern shown in FIG. 13A is an example of one possible IOL axis implantation pattern. Many other axis signaling patterns can be used for the same purpose without departing from the scope of the present invention.

Shown in FIG. 13B is another alternative embodiment, as a mode of example only, where marks instead of enclosed incisions are used as axis references for a surgeon. Two diametrically opposed axis marks 925 and 926 are shown in capsule 402 oriented across the center of incision 400. These marks allow a surgeon to trace with precision an imaginary line 604 to accurately rotationally deliver a toric IOL 408. Processor 300 can be programmed through UI 306 to deliver the UF light source pulses in a capsule marking modality, rather than a capsule incision modality. These marks can be effectively made using reduced UF light energy, reduced UF pulse duration, changing the wavelength of the UF light, reducing the number of treatment spots, reducing the number of treatment iterations in a same region, and/or increasing the spacing between the UF light treatment spots, or any combination of the aforementioned parameters. In this way a selected region of the capsule is visibly marked by changes in the tissue structure and micro-bubble entrapment in the crevices created by the laser spots within the capsule. The marked capsule tissue while visible to a surgeon shows no reduction in resistance to tearing of significance when using proper parameters. As a mode of example, using a UF light power of 2 uJ with spacing of 50 microns in a pattern covering a square area of 350×350 microns produces a visible capsule mark without cutting or weakening of the capsule. In this example a single pair of diamond shaped laser marks with each side measuring 0.35 mm is shown, in a way that each mark corner is spaced 0.25 mm along parallel lines drawn across the meridian corresponding to the axis of both opposing marks. In this way marks allow precise rotational placement of an axis-marked IOL and also provide a good dimensional reference for IOL centration and other purposes, in this case 0.25 mm and 0.5 mm.

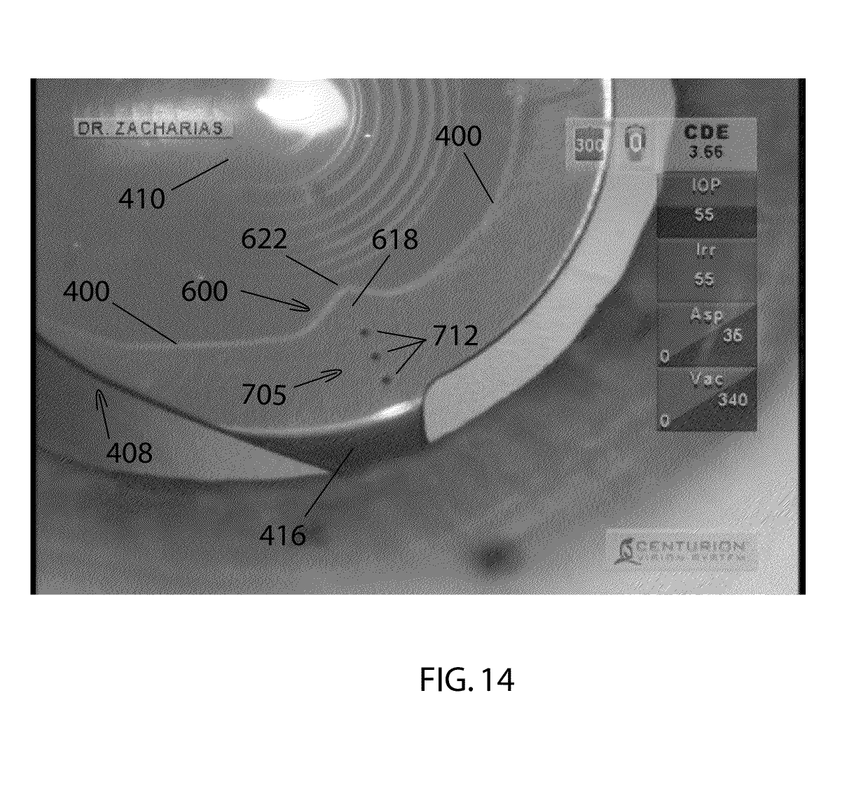

FIG. 14 is a real life picture of an eye 68 with properly placed toric IOL 408 inside capsule 402. Alignment marks 618 and 712 radially coincide indicating a proper match between desired axis of IOL implantation as indicated by surgeon guiding incision features 600 and IOL axis features 705. The centripetal apex 622 of curvilinear flap 618 is perfectly in-line with a line fitting along IOL marks 712. For proper IOL rotary alignment to capsule 402, diametrically opposed features 600 and 705 must be aligned in a similar fashion (not shown). As can be observed, a surgeon has IOL reference marks 705 and 600 simultaneously in focus, with minimal parallax, with perfect guidance to desired centration and rotational IOL positioning.

Opposing axis guiding features 705 and 600 are preferably diametrically aligned passing through the optical center of the IOL 408 and of capsule incision 400. While this disposition of axis marking features is desirable, it can change to different location in the IOL and/or in the capsule incision, for example, passing through the center of the capsular bag, or other eye landmark or IOL landmark without departing from the scope of the present invention.

The present invention is not limited to the embodiment described above and illustrated herein, but encompasses any and all variations falling within the scope of the appended claims. For example, references to the present invention herein are not intended to limit the scope of any claim or claim term, but instead merely make reference to one or more features that may be covered by one or more of the claims. All the optical elements downstream of scanner 50 shown in FIGS. 1, 3 and 4 form a delivery system of optical elements for delivering the beam 6, 114 and 202 to the target tissue. Conceivably, depending on the desired features of the system, some or even most of the depicted optical elements could be omitted in a delivery system that still reliably delivers the scanned beams to the target tissue.

Enclosed main incision based axis indicating features can be replaced by UF laser marks/incisions in the peripheral capsule tissue. Steep corneal meridian axis marks or features could be replaced or supplemented by flat corneal meridian marks or features matching the corresponding IOL convention for astigmatism alignment. IOL calculation software can recommend implantation of the IOL in an axis matching an orientation that is different to the steepest axis of the eye cornea, for example, to compensate for surgically induced astigmatism, incision location, surgeon calibration factor, etc. In such case the operator of system 2 will program accordingly to set the axis indicating incisions, features or marks at an angle that may not coincide with the steep axis of astigmatism of the patient's cornea. Femtosecond LASER could be replaced by other similarly acting UF light source. Inward capsule incision deformations as features for alignment between corneal astigmatism and IOL astigmatism can be replaced by a plurality of different marking features such as outward capsule incision deformations, flaps, intrusions, extrusions as long as incision resistance to elongation and deformation is not compromised. Capsule axis marking features can also be used to rotationally position non-toric IOLs with axis relevant conditions, such as for example radially segmented multifocal IOLs. UF laser marks/incisions can be placed using system 2 in other surgeon observable eye tissues such as the cornea without departing from the scope of the present invention.

1. (canceled)

2. (canceled)

3. (canceled)

4. A method of inserting an intraocular lens in a patient's eye, comprising: detecting traceable eye landmarks; detecting the astigmatism of the cornea; registering the axis of the corneal astigmatism to the traceable eye landmarks; generating a light beam; deflecting the light beam using a scanner to form an enclosed treatment pattern and a registration pattern peripheral to the enclosed treatment pattern which is linkable to the recorded eye landmarks registered to the corneal astigmatism axis; and placing an intraocular lens within the enclosed incision, wherein the intraocular lens has a lens astigmatism axis visible registration feature that that the surgeon aligns with the visible registration feature of the peripheral incision/marks.

5. The method of claim 4 wherein the registration feature peripheral of the enclosed incision is an opposing pair of marks/incisions both fitting into a straight line describing the astigmatism axis.

6. The method of claim 4 wherein the registration feature peripheral of the enclosed incision is a plurality of opposing pairs of marks/incisions each pair fitting into parallel straight lines describing the astigmatism axis.

7. (canceled)

8. A method to produce marks in eye tissue visible by a surgeon consisting in:
a) defining a spatial mark shape and pattern and location;
b) generating a light beam with adjusted parameters to produce marks in tissue;
c) deflecting the light beam using a scanner to deliver light pulses according to the shape, pattern and selected location.

9. The method of claim 8 to produce marks visible by a surgeon in the lens capsule of the eye.

10. The method of claim 8 to produce marks visible a surgeon in the cornea of the eye.

11. A method for correct alignment of an astigmatism-correcting intraocular lens inside the lens capsule of an eye comprising:
a) selecting a preferred axis in a lens capsule to which the axis of an astigmatism-correcting intraocular lens astigmatism should be matched;
b) generating a light beam;
c) deflecting the light beam using a scanner to form a main enclosed treatment pattern and peripheral enclosed incisions/marks indicative of said preferred axis for intraocular lens orientation;
d) that includes a feature visible to a surgeon indicative of said preferred axis for intraocular lens orientation;
e) delivering the main enclosed treatment pattern including visible peripheral enclosed incisions/marks to target tissue in the patient's eye to form an enclosed incision including peripheral enclosed incisions/marks;
f) placing within the enclosed incision an intraocular lens, wherein the intraocular lens has visible axis marks;
g) positioning said intraocular lens inside said capsule until said intraocular lens axis marks relate to said visible capsule peripheral incisions/marks in a way that the intraocular lens axis marks are properly aligned with said preferred axis.
